(19) United States
(12) Patent Application Publication
(10) Pub. No.: US 2017/0245881 A1
(43) Pub. Date: Aug. 31, 2017

(54) AMPUTATOR USING ULTRASONIC WAVES FOR AMPUTATING AND ULTRASONIC SURGICAL APPARATUS PROVIDED WITH SAME

(71) Applicant: Ligasonix, Inc., New York, NY (US)

(72) Inventor: Eung Kook KIM, Seocho-gu (KR)

(21) Appl. No.: 15/122,399

(22) PCT Filed: Feb. 28, 2014
(86) PCT No.: PCT/KR2014/001709

§ 371 Date: Jan. 30, 2017

(51) Int. Cl. A61B 17/32

(52) U.S. Cl. CPC A61B 17/320092; A61B 2017/320072

(57) ABSTRACT

Disclosed is an amputator, which is provided at one end of a transmitting rod connected to a vibrator generating ultrasonic waves, for amputating a surgical site by means of ultrasonic waves transmitted from the transmitting rod, the amputator comprising: an amputating side which comes in contact with the surgical site; and a direction switching means, which has a depression in one part thereof from a point corresponding to a vibration node, for concentrating the ultrasonic waves on the amputating side, the ultrasonic waves being transmitted at an angle toward the amputating side along the lengthwise-direction.

### TECHNICAL FIELD

The present invention relates to an amputator using ultrasonic waves and an ultrasonic surgical apparatus provided with the same, and more particularly, an amputator using ultrasonic waves that can amputate faster and more effectively by regulating the intensity of the ultrasonic waves applied to the amputating side and an ultrasonic surgical apparatus using the same.

### BACKGROUND ART

Recently, surgical apparatuses using ultrasonic waves are being developed. Among them, the surgical apparatuses similar with harmonic scalpel are the most representative.

The surgical apparatuses having such shape normally comprises: a gun-shaped handle unit gripped in the operators hand; an amputator provided in front of the handle unit for amputating a surgical site using ultrasonic waves transmitted from a vibrator provided in the handle unit; and a jaw provided at a position facing with the amputator for gripping a surgical site together with the amputator.

The amputator in accordance with the present invention is devised for amputating a surgical site using ultrasonic waves received from an ultrasonic wave generator. While the amputators can be configured in various ways, and FIG. 1 shows an ordinary, conventional amputator.

As shown in FIG. 1, a typical amputator of conventional configuration has a minimized size (volume) in order to concentrate ultrasonic waves on the amputating side, however, reducing size (volume) only can result in uneven distribution of ultrasonic waves applied to an amputating side according to the point of the reduction of the size (volume).

### DETAILED DESCRIPTION OF THE INVENTION

### Technical Objects

An aspect of the present invention is to provide an amputator using ultrasonic waves provided with a direction conversion means that can concentrate ultrasonic waves on the amputating side by more even distribution to accomplish faster and more effective amputating and an ultrasonic surgical apparatus using the same.

The present invention is not restricted to the technical objective set forth above. The above and other aspects of the invention not described herein will become apparent to those skilled in the art to which the invention pertains by referencing the detailed description of the invention below.

### Means for Achieving the Technical Object

To solve above described problem, the amputator using ultrasonic waves in accordance with an embodiment of the present invention is provided on one end of a transmission rod connected to a vibrator, and amputates a surgical site using the ultrasonic waves transmitted through the transmission rod. A portion of the amputating side contacting with a surgical site and from the point corresponding with a vibration node are concaved, forming a certain slope angle towards the amputating side along the lengthwise direction to concentrate ultrasonic waves on the amputating side, which constitutes a direction conversion means.

Here, the direction conversion means can be characterized by a concave whose depth increases according to the distance from the point of vibration node.

The direction conversion means is characterized by having a predetermined length and a first slope formed in the direction meeting with a virtual line extending in the lengthwise direction of the transmission rod and a second slope formed by being extended from one end in the lengthwise direction of the first slope and parallel with a virtual line extending the lengthwise direction of the transmission rod.

The first and the second slopes are formed in straight lines and connected at a predetermined angle.

In addition, the first slope can be characterized by the portion formed by the second slope being extended is formed in a curved line.

In addition, the direction conversion means can be characterized by having the concave in an angled shape.

In addition, the direction conversion means can be characterized by being provided in a pair, arranged left-right symmetrical pivoting up-down direction, when positioned with the amputating side above, and sloped in the same direction.

In addition, the maximum thickness in the vertical direction is not changed when positioned with the amputating side above.

The ultrasonic surgical apparatus devised to solve above described problems in accordance with another aspect of the present invention comprises: a vibration device for generating ultrasonic waves; a transmission rod formed in a cylindrical bar to transmit ultrasonic waves generated by a vibrator coupled to one end to an amputator formed by extending from an opposite end; An amputator provided on one end of a transmission rod connected to a vibrator, and amputates a surgical site using the ultrasonic waves transmitted through the transmission rod, and a portion of the amputating side contacting with a surgical site and from the point corresponding with a vibration node are concaved, forming a certain slope angle towards the amputating side along the lengthwise direction to concentrate ultrasonic waves on the amputating side, which constitutes a direction conversion means; A rod cover surrounding the transmission rod using a plurality of vibration nodes form in the process of transmitting ultrasonic waves through the transmission rod as the connecting points; and a jaw coupled with one end of the rod cover in a tiltable manner, at a position facing with the amputator to grip the surgical site by mating with the amputator.

### Effect of the Invention

An aspect of the present invention is to provide an amputator using ultrasonic waves provided with a direction conversion means that can concentrate ultrasonic waves on the amputating side by more even distribution to accomplish faster and more effective amputating and an ultrasonic surgical apparatus using the same.

The effect of the present invention is not restricted to those set forth above. The above and other aspects of the invention will become apparent to those skilled in the art to which the invention pertains by the description of the claims.

### BRIEF DESCRIPTION OF THE DRAWING

FIG. 1 is a schematic, perspective, view of a conventional amputator,

FIG. 2 is an exemplary ultrasonic surgical apparatus using an amputator using ultrasonic waves in accordance with an embodiment of the present invention,

FIG. 3 is a schematic perspective view of the configuration of the amputator and transmission rod of the surgical apparatus of FIG. 2,

FIG. 4 is a perspective view of the amputator and transmission rod of the surgical apparatus of FIG. 2 in a different angle of view,

FIG. 5 is a side view of the amputator and transmission rod of the surgical apparatus of FIG. 2,

FIG. 6 is a cross-sectional view taken from line A1 of FIG. 5,

FIG. 7 is a cross-sectional view taken from line A2 of FIG. 5,

FIG. 8 is a cross-sectional view taken from line A3 of FIG. 5,

FIG. 9 is a cross-sectional view taken from line A4 of FIG. 5,

FIG. 10 is a cross-sectional view taken from line A5 of FIG. 5,

FIG. 11 is a cross-sectional view of an amputator having a round-shape sunk,

FIG. 12 is a schematic diagram showing the surgical apparatus utilizing ultrasonic waves of FIG. 2 approaching a surgical site,

FIG. 13 is a schematic diagram showing the surgical apparatus utilizing ultrasonic waves of FIG. 11, gripping and amputating a surgical site,

FIG. 14 is a view showing the deformed shape of the direction conversion means of the amputator of FIG. 2,

FIG. 15 is a view showing another deformed shape of the direction conversion means of the amputator of FIG. 2, and

FIG. 16 is a view showing the direction conversion means of the amputator of

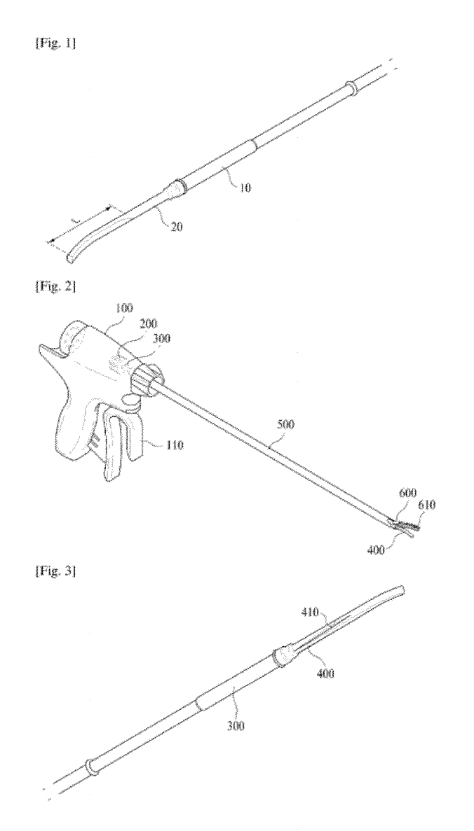

FIG. 2, having different left and right side slope angles.

### NUMBERING SCHEME OF THE MAJOR PARTS OF THE DRAWINGS

100: handle unit
200: vibrator
300: transmission rod
400: amputator
410: direction conversion means
412: first slope portion
414: second slope portion
420: amputating side
500: rod cover
600: jaw

### MODE FOR CARRYING OUT THE INVENTION

An embodiment of the present invention is described hereinbelow by referring to the accompanying drawings. In the description of the present invention, known functions and/or configuration can be omitted in order to clarify the spirit of the present invention.

In the description of the present invention, the terms referring to directions, i.e., up, down, or front, rear, are used solely to help those skilled in the art understand the present invention more clearly. As such, such directions are on a relative basis and shall not be interpreted to restrict the scope of the right of the present invention.

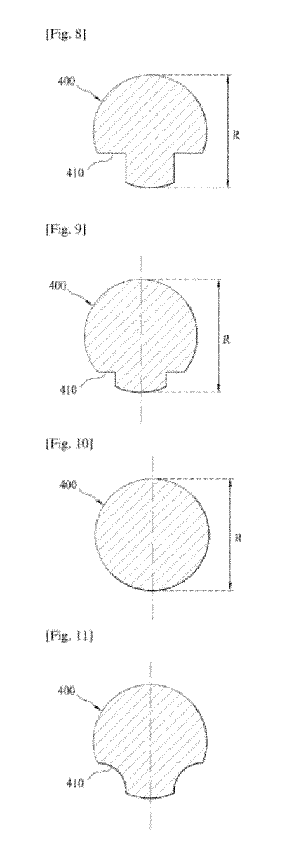

As shown in FIGS. 2 through 16, the embodiment of an amputator 400 using ultrasonic waves and a surgical apparatus using the same comprises: a handle unit 100 gripped in a hand of an operator, a vibrator 200, a transmission rod 300, an amputator 400, a rod cover 500 and a jaw 600.

The handle unit 100 is provided in a gun-shape for being held in a hand of an operator, comprising: a vibrator 200 inside for generating vibration, a transmission rod 300 connected with the vibrator 200 and a rod cover 500 connected in forward direction. In addition, an additional operating means 110 is provided to control the wavelength, amplitude and frequency of the ultrasonic waves generated by the vibrator 200.

The vibrator 200 generates ultrasonic waves by receiving electric signals from input terminals. (However, the electric circuit and wiring for transmitting the signals between the vibrator 200 and the input terminals are not indicated in the drawings.)

The frequency of the ultrasonic waves generated by the vibrator 200 varies by the conditions of the transmission rod 300 and amputator 400 which will be described later. That is, the ultrasonic waves generated by the vibrator 200 can be adjusted by the configuration and material of the transmission rod 300 and/or design of the gain steps and operating length of the amputator 400.

In addition, the wavelength, amplitude and frequency of the ultrasonic waves generated by the vibrator 200 can be controlled with the operating means 110 taking the surgical site S to be amputated into consideration.

For example, if the surgical site S is a thick tissue or containing a blood vessel, the intensity of the ultrasonic waves should be adjusted to be higher, or the intensity can be controlled to be lower to avoid hindrance of the byproducts of amputation to the operation.

Meanwhile, the transmission rod 300 connects the vibrator 200 and amputator 400 to transmit the ultrasonic waves generated by the vibrator 200 to the amputator 400.

The transmission rod 300 in accordance with an embodiment of the present invention is provided in a long, rod-shape whose one end and the opposite end are connected to the vibrator 200 described earlier and amputator 400 described later, respectively.

While not shown in the drawings, the transmission rod 300 can be configured in a long, thin, cylindrical bar so that the amputator 400 can be inserted in a hole formed on a trocar used in laparoscopy to reach a surgical site S easily.

In addition, the length of the transmission rod 300 can be provided sufficiently for laparoscopy, and to allow repetition of the vibration 200 nodes (the points where the intensity of ultrasonic wave is substantially zero) and vibration anti-node (a concept opposite to the vibration node, the points where the intensity of ultrasonic wave is the highest) by a plurality of numbers.

In addition, while the transmission rod 300 can be made with various materials, titanium material would be preferable for efficient transmission of the ultrasonic waves generated in the vibrator 200 as described earlier.

In the present embodiment, the transmission rod 300 is formed in a long cylindrical bar shape, a portion of which is connected inside of the handle unit 100 and the opposite portion protrudes out forward.

The amputator 400 for cutting a surgical site S utilizing ultrasonic waves is provided on one end of the transmission rod 300 to receive the ultrasonic waves generated by the vibrator 200 to cut a surgical site S.

The amputator 400 in accordance with the present invention can be formed by being extended from one end of the transmission rod 300 described earlier, and can cut a surgical site S by pressing and gripping the surgical site S together with the jaw 600, to be described later, provided on the opposite side.

Since the amputator 400 operates utilizing ultrasonic waves, it does not have a sharp edge, different from other ordinary cutting devices. That is, the amputator 400 is designed to cut a surgical site S using ultrasonic waves, thus, it is effective when amputating a surgical site S through which a blood vessel or vessels pass.

Since the amputator 400 should be able to amputate a surgical site S utilizing the ultrasonic waves transmitted through the transmission rod 300, it should have a gain step (indicating that the actual ratio of amplification is 1 or higher).

For a means to provide the amputator 400 with a gain step, the shape and jaw 600 of the amputator 400 can be diversified, In accordance with an embodiment of the present invention, the lateral cross-sectional area of the amputator 400 is reduced to be smaller than that at the vibration node so that the energy of the ultrasonic waves can be amplified according to the ratio of the cross-sectional area. Accordingly, when the amputator 400 is formed by being extended from the transmission rod 300, a portion of the round cross-sectional area is flattened to reduce the area and to form a gain step.

In the formation of the amputator 400, one thing that is as important as the gain step is active length.

That is, even the amputator 400 amplifies the energy of ultrasonic waves with gain step, if the active length is too short, i.e., if the intensity is not sufficient for amputating a surgical site S, the apparatus would be useless for a user who desires to amputate a surgical site S.

To secure sufficient active length, the amputator 400 in accordance with an embodiment of the present invention is provided with an amputating side 420 contacting with a surgical site S, and direction conversion means 410, a portion of which is concaved from the point of vibration node and sloped lengthwise in the direction of the amputating side 420 to concentrate transmitted ultrasonic waves to the amputating side 420.

Here, the direction conversion means 410, when positioned with the amputating side 420 above, is formed on the side in the lengthwise direction of the amputator 400 with the concave depth increasing gradually according to the distance from the point of vibration node.

As shown in FIG. 5, the direction conversion means 410 is formed crossing a virtual extension line X in the lengthwise direction of the transmission rod 300, sloped upward in the direction of the amputating side 420.

As the direction conversion means 410 is formed with concave and sloped upward in the direction of the amputating side 420, the ultrasonic waves transmitted through the transmission rod 300 is concentrated towards the amputating side 420 by the direction conversion means 410.

In the present embodiment in accordance with the present invention, the direction conversion means 410 are provided in a pair arranged symmetrically left and right, centering a vertical axis, when the amputating side 420 is in the upper position, in order for effective transmission of the ultrasonic waves transmitted through the transmission rod 300 to the amputating side 420. Different from the drawing, the amputator may be provided with a single direction conversion means 410

The direction conversion means 410 in accordance with the present invention are sunk from the bottom of the point corresponding with a vibration node and sloped upwards up to the cross point with the extension line X, and are formed along a portion of the length of the amputator 400.

Here, the angle between the extension line X and the direction conversion means 410 can be varied according to the intensity (frequency, amplitude) of the ultrasonic waves and the material of the amputator 400. In the present embodiment in accordance with the present invention, the slope angle of the direction conversion means 410 can be in the range of between from about 2 to about 7 degrees.

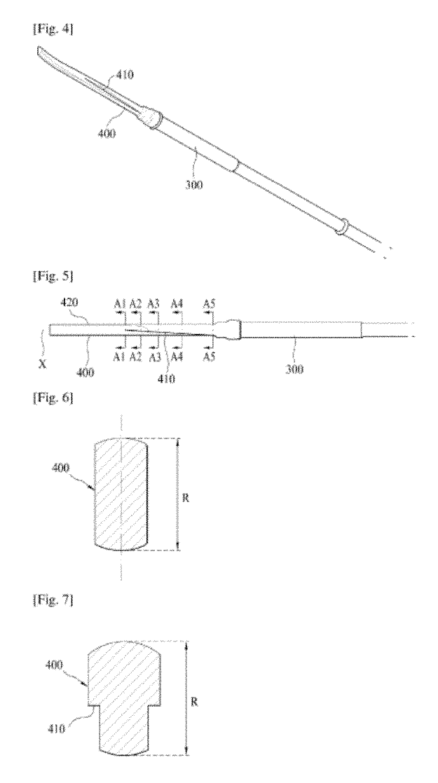

The shape of the direction conversion means 410 in the present embodiment in accordance with the present invention is discussed in further details. As shown in FIGS. 6 through 10, the direction conversion of the amputator 400 means 410 is concaved from the points corresponding with vibration nodes and the depth of the concave becomes deeper as the distance from the vibration node increases.

The purpose of this design is to generate gain steps by reducing the cross-sectional area of the amputator 400.

Here, while the direction conversion means 410 is sloped upward from the vibration nodes along the lengthwise direction of the amputator 400, however, the slope ends before arriving at the amputating side 420.

In the present embodiment, one end of the direction conversion means 410 extends up to the center point with reference to the vertical length R of the amputator 400. However, the actual configuration can be changed according to the intensity of the ultrasonic waves or the material of the amputator 400.

As shown in the figures, the amputator 400 is formed with direction conversion means 410 in a concaved shape that reduces the cross-sectional area of the amputator 400 in the lengthwise direction, the maximum vertical thickness of the amputator 400 is not changed.

Accordingly, when the jaw 600 pushes towards the amputating side 420 as described later, the deflection of the amputator 400 can be minimized protecting the amputator 400 from being damaged.

Meanwhile, the direction conversion means 410 in accordance with the present invention is formed as a concave in an angled shape on the amputator 400. As a result, the direction conversion means 410 can further concentrate the ultrasonic waves transmitted through the transmission rod 300.

As shown in FIG. 11, it the concaved point of the direction conversion means 410 is curved, the concentration effect of the ultrasonic waves transmitted to the amputator 400 resulting in reduced intensity of the ultrasonic waves concentrated on the amputating side 420.

As described above, the amputator 400 in accordance with the present invention is concaved by a certain length in the lengthwise direction and provided the direction conversion means 410 formed towards the amputating side 420 so that the ultrasonic waves can be transmitted to the amputating side 420 with an even intensity.

The rod cover 500 surrounds the transmission rod 300 to protect the ultrasonic waves transmitted through the transmission rod 300. As such, the rod cover 500 in accordance with an embodiment of the present invention is a long, hollow rod, allowing the transmission rod 300 to penetrate through the hollow portion.

In addition, the opposite end is coupled and fixed with the handle unit 100 described earlier, and the amputator 400 penetrates and exposed outwards through the one end.

The rod cover 500 in accordance with an embodiment of the present invention has a length corresponding with the length of the transmission rod 300 described earlier, the opposite end is coupled with the handle unit 100 and the amputator 400 protrudes out from the hollow of the rod cover.

While the rod cover 500 is preferably not contacting with the transmission rod 300, however, it may be necessary to be connected if the transmission rod 300 is long. Here, the connection point is preferably be made on a plurality of the vibration nodes on the transmission rod 300 formed by the ultrasonic waves.

This is because, since the intensity of the ultrasonic waves is not zero at any other points than the vibration nodes, if the transmission rod 300 and rod cover 500 are connected at any other points than the vibration nodes, the energy of the ultrasonic waves being transmitted through the transmission rod 300 is transmitted to the rod cover 500 and lost (wasted).

That is, though not shown, the rod cover 500 and transmission rod 300 in accordance with an embodiment of the present invention can be configured to contact with each other partially along on the lengthwise direction.

The jaw 600, positioned facing with the amputator 400 to grip a surgical site S by adhering to the amputator 400. In the present invention, the jaw is selectively tilted so that one side can contact with the amputator 400.

If without the jaw 600, it is difficult to amputate a surgical site S because the surgical site S is not backed up when pushed by the amputator 400 for amputating. That is, the jaw 600 assists 600 the amputator 400 for amputating a surgical site S.

The jaw 600 may be formed by being integrated with the transmission rod 300, however, such configuration can hinder the transmission of ultrasonic waves to the amputator 400. Therefore, it is preferably coupled with an end of the rod cover 500.

In addition, it can be coupled with the rod cover 500 in a tiltable manner for easier gripping and releasing of a surgical site S.

In addition, the jaw 600 can be provided grooves 610 forms by predetermined intervals on the inner side facing one side of the amputator 400 in order to prevent the surgical site S from being released from between the amputator 400 and the jaw.

Here, the groove 610 can be tilted towards the lengthwise direction of the jaw 600, with reference to the direction perpendicular with the lengthwise direction of the jaw 600.

That is, if the lengthwise direction of the jaw 600 is defined by a direction parallel with the line connecting a vibration mode N1 and an anti-vibration node A1, and let the slope of a line perpendicular with the lengthwise direction is zero (0), the grooves 610 can be sloped by a certain angle in the lengthwise direction of the jaw 600

That is, if the certain angle is small, the grooves 610 are formed perpendicular to the lengthwise direction of the jaw 600, and if the angle is large, the grooves 610 are formed in the lengthwise direction of the jaw 600.

The surgical apparatus utilizing ultrasonic waves and the amputator 400 are described in detail hereinabove. The gripping of the surgical site S by the surgical apparatus utilizing ultrasonic waves in accordance with an embodiment of the present invention is described in detail below.

As shown in FIG. 12, the amputator 400 in accordance with an embodiment of the present invention approaches close to a surgical site S without contacting with each other and with the jaw 600 being tilted. Here, a portion of the surgical apparatus utilizing ultrasonic waves has already been inserted in the body of an operatee, and the surgical site S refers to a portion of the body which is the target of the operation.

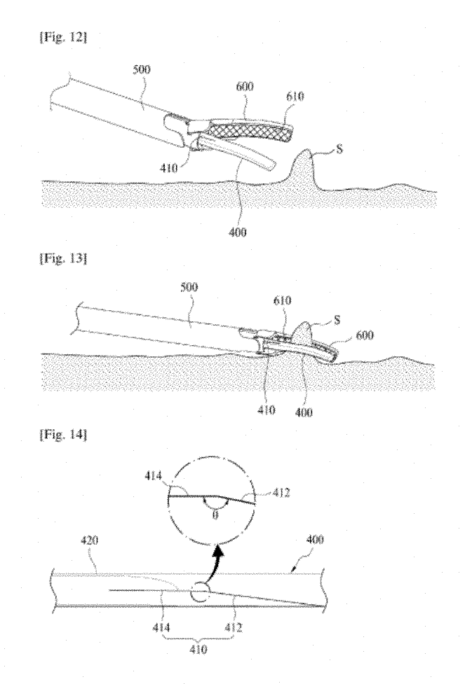

After inserted into the body of the operate, the amputator 400 and jaw 600 grip the surgical site S or part to be cut off, as shown in FIG. 13. At this time, the operator tilts the jaw 600 by using the operating means 110, so that the amputator 400 and one side of the jaw 600 contact with and grip the surgical site S.

Then, the operator starts the vibrator 200 to amputate the surgical site S.

With the above described method, the ultrasonic surgical apparatus in accordance with an embodiment of the present invention can perform amputating a surgical site S faster and more effectively.

Referring to FIGS. 14 and 15, the variants of the amputator 400 in accordance with the present invention are described below.

A variant of the amputator 400 in accordance with the present invention is provided with the direction conversion means 410 which comprises a first slope portion 412 and a second slope portion 414.

The first slope portion 412 is sloped in the direction to the crossing point with a virtual extension line X in the lengthwise direction of the transmission rod 300. The second slope portion 414 is formed by extending from one end of the first slope portion 412 in the lengthwise direction in parallel with the extension line X.

That is, the first slope portion 412 and the second slope portion 414 are formed in long lines meeting with each other in the lengthwise direction, having different angles of slope.

Since the direction conversion means 410 comprise a first slope portion 412 and a second slope portion 414 having different slope angles, the ultrasonic waves transmitted to the amputator 400 can be effectively concentrated on the amputating side 420.

As shown in FIG. 14, the first slope portion 412 and a second slope portion 414 are formed in a straight line which can meet with each other at a predetermined angle. Here, the first slope portion 412 can be formed at the point formed by extension of the second slope portion 414 at a predetermined angle.

In addition, as shown in FIG. 15, the first slope portion 412 can be formed at the point formed by extension of the second slope portion 414 in a curved line.

That is, while the first slope portion 412 and the second slope portion 414 are formed with different slope angle, the two can cross each other in straight or curved lines.

While the second slope portion 414 of the present embodiment is parallel with the extension line X, the relation between the two lines is not limited to parallelism but can form any angle. In addition, it is be obvious that the first slope portion 412 and the second slope portion 414 can be formed in curved lines, not the straight line described in the present embodiment.

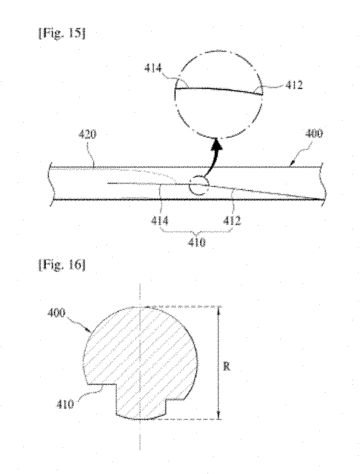

The variants of the direction conversion means 410 formed on the amputator 400 are described by referring to FIG. 16 below.

FIG. 16 is a cross-sectional view of the direction conversion means 410 formed with different slope angles on the amputator 400 of FIG. 2.

As shown in, the direction conversion means 410 can be formed on the amputator 400 with different angles on the left and right sides when placed under the amputating side 420.

More particularly, referring to FIG. 16 showing the cross-sections along the lengthwise direction of the amputator 400, the direction conversion means 410 can be formed on the amputator 400 with different angles on the left and right sides to transmit the ultrasonic waves transmitted from the transmission rod 300 to the amputating side 420.

Here, since a pair of direction conversion means 410 are provided with different slope angles, the intensity of the ultrasonic waves transmitted to the amputating side 420 is changed.

More particularly, by providing the direction conversion means 410 with different slope angles, the intensity of the ultrasonic waves transmitted to the amputating side 420 can be adjusted by the materials of the amputator 400 and transmission rod 300 and the intensity of the ultrasonic waves generated in the vibrator 200.

While an embodiment of the present invention is described by referring to a harmonic scalpel, it would be obvious that the present invention can be applied to other types of surgical instruments utilizing ultrasonic waves, and further, other medical systems such as computer-integrated robotic surgery systems.

From the foregoing it is believed that those skilled in the pertinent art will recognize the meritorious advancement of this invention and will readily understand that while the present invention has been described in association with a preferred embodiment thereof, and in the accompanying drawings, numerous changes and modifications may be made therein by those skilled in the art without departing from the spirit and scope of this invention. Therefore, the modifications and changes shall not be interpreted independently from the technical spirit and point of view, and the modified embodiments shall be interpreted to be within the scope of the present invention.

What is claimed is:

1. An amputator provided at one end of a transmission rod connected to a vibrator that generated ultrasonic waves for amputating a surgical site using the ultrasonic waves transmitted from the transmission rod, comprising:
an amputating side contacting with the surgical site; and direction conversion means formed as a depression from a point corresponding with a vibration node, having slope angles along the lengthwise direction towards the amputating side to concentrate the ultrasonic waves on the amputating side.

2. The amputator of claim 1,
wherein the direction conversion means
are characterized by a depression whose depth increases according to the distance from the point of vibration node.

3. The amputator of claim 2,
wherein the direction conversion means comprises:
a first slope formed in the direction meeting with a virtual line extending in the lengthwise direction of the transmission rod; and a second slope formed by being extended from one end in the lengthwise direction of the first slope and parallel with a virtual line extending the lengthwise direction of the transmission rod.

4. The amputator of claim 3,
wherein the first and the second slopes are formed in straight lines and connected at a predetermined angle.

5. The amputator of claim 3,
wherein the first slope is characterized by the portion formed by the second slope being extended is formed in a curved line.

6. The amputator of claim 1,
wherein the direction conversion means is characterized by,
having an angle-shaped depression.

7. The amputator of claim 1,
wherein the direction conversion means is characterized by;
being provided in a pair, arranged left-right symmetrical pivoting up-down direction, when positioned with the amputating side above, and sloped in the same direction.

8. The amputator of claim 1,
wherein the maximum thickness in the vertical direction is not changed when positioned with the amputating side above.

9. A surgical apparatus comprising:
a vibrator for generating ultrasonic waves;
a transmission rod formed in a cylindrical bar to transmit ultrasonic waves generated by a vibrator coupled to one end to an amputator formed by extending from an opposite end;
an amputator provided on one end of a transmission rod connected to a vibrator, and amputates a surgical site using the ultrasonic waves transmitted through the transmission rod, and a portion of the amputating side contacting with a surgical site and from the point corresponding with vibration node are concaved, forming a certain slope angle towards the amputating side along the lengthwise direction to concentrate ultrasonic waves on the amputating side, which constitutes a direction conversion means;
A rod cover surrounding the transmission rod using a plurality of vibration nodes form in the process of transmitting ultrasonic waves through the transmission rod as the connecting points; and
a jaw coupled with one end of the rod cover in a tiltable manner, at a position facing with the amputator to grip the surgical site by mating with the amputator.
